(19) United States
(12) Patent Application Publication
(10) Pub. No.: US 2024/0197617 A1
(43) Pub. Date: Jun. 20, 2024

(54) ANTI-AGING COSMETIC COMPOSITION BASED ON RESVERATROL AND MAGNOLIA

(71) Applicants: Tomcat International Limited, London (GB); PRESIDENT AND FELLOWS OF HARVARD COLLEGE, Cambridge, MA (US)

(72) Inventors: Mathilde THOMAS, Paris (FR); Bertrand THOMAS, Paris (FR); David SINCLAIR, Chestnut Hill, MA (US)

(21) Appl. No.: 17/788,895

(22) PCT Filed: May 10, 2022
(86) PCT No.: PCT/EP2022/062610

§ 371 Date: Jun. 24, 2022

(30) Foreign Application Priority Data
May 10, 2021 (FR) 21/04931

(51) Int. Cl. A61K 8/9789; A61Q 19/08

(52) U.S. Cl. CPC A61K 8/9789; A61Q 19/08; A61K 2800/78

(57) ABSTRACT

An anti-aging cosmetic composition including resveratrol and/or a derivative of resveratrol in combination with honokiol and/or magnolol. These molecules can be obtained from plant extracts. Both honokiol and magnolol are present in magnolia bark. A cosmetic treatment method is related including the application to the skin or skin appendages of the cosmetic composition.

### TECHNICAL FIELD

The present disclosure concerns a cosmetic composition for combating the signs of aging of the skin and skin appendages in order to improve their appearance, as well as the cosmetic uses of such a composition, such as a cosmetic treatment method implementing it.

### BACKGROUND

The aging phenomena, whether of intrinsic or extrinsic origin, are normal natural phenomena. But there is still a need for a large number of people to improve the appearance of their skin or their skin appendages and to reduce or delay the signs associated with their aging.

Deoxyribonucleic acid (hereafter abbreviated «DNA») methylations are environmentally induced epigenetic modifications that alter cytosines to 5-methylcytosines (hereafter abbreviated «5-mC») on dinucleotides CpG (or otherwise hereinafter referred as «CpG sites»), namely «cytosine-guanine» nucleic base sequences. In this regard, it should be noted that the notation «CpG» is an abbreviation for «cytosine-phosphate-guanine» to be distinguished from the abbreviation «CG» which designates a pair of nucleic bases on two distinct strands of DNA. In the genomes, the CpG sites have a specific distribution in the form of islands of CpG sites in which their concentration is high and they then act in the regulation of the gene expression.

Thus, these epigenetic modifications occur mainly in the islands of CpG sites, which are regions rich in CpG sites mainly located at the level of gene promoters. They are involved in the regulation of gene expression since the methylation of a gene induces the repression of its expression.

These methylations are carried out by enzymes which are DNA methyltransferases.

The demethylation, for its part, passes through intermediate forms such as 5-hydroxymethylcytosine (hereinafter abbreviated «5-hmC»).

The generation of 5-hmC from 5-mC is catalysed by enzymes which are 5-mC hydroxylases or otherwise hereinafter referred as «TET enzymes»((«TET» being the acronym of «ten-eleven translocation enzymes»). In other words, these enzymes hydroxylate 5-mC to 5-hmC.

Yet, several links have been established between cellular aging and DNA methylation. Indeed, it seems that, although at the global level DNA methylation tends to decrease, at the specific level (and in particular at the level of gene promoters), an hypermethylation has been observed. These hypermethylations could be linked to the alteration of the expression of key genes during aging.

This is why, at the cutaneous level, it is advantageous to fight against the hypermethylation of age-related genes by stimulating the activity of TET enzymes.

In this regard, the publication entitled «Honokiol inhibits ultraviolet radiation-induced immunosuppression through inhibition of ultraviolet-induced inflammation and DNA hypermethylation in mouse skin» by Prasad et al., published on May 10, 2017 in Scientific reports, 7: 1657, relates a study carried out on a mouse which showed that honokiol, namely a molecule present in magnolia bark, stimulated the activity of TET enzymes.

The magnolia is a tree of the magnoliaceae family and is native to China. It is a medicinal plant belonging to the Chinese pharmacopoeia. The bark of this tree is rich in lignans (that is to say phenolic compounds) including honokiol and magnolol which are active molecules.

The honokiol has the following chemical formula (1):

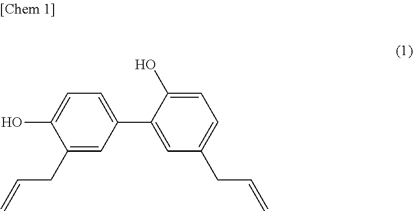 [SMILES: C=CCc1ccc(O)c(-c2ccc(O)c(CC=C)c2)c1]

The magnolol has the following chemical formula (2):

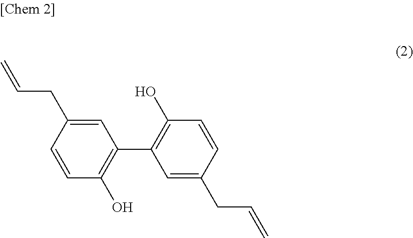 [SMILES: C=CCc1ccc(O)c(-c2cc(CC=C)ccc2O)c1]

It is known to use magnolia bark extracts which contain approximately 5% by mass of these two honokiol and magnolol molecules in cosmetic compositions for their anti-aging, anti-acne and soothing properties.

Moreover, the molecule of resveratrol, namely 3,5,4′-trihydroxystilbene, is known, which exists in both cis and trans isomeric forms.

The cis-resveratrol has the following chemical formula (3):

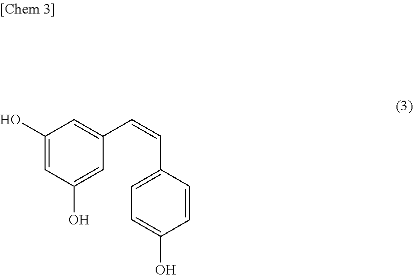 [SMILES: Oc1ccc(/C=C\c2cc(O)cc(O)c2)cc1]

The trans-resveratrol has the following chemical formula (4):

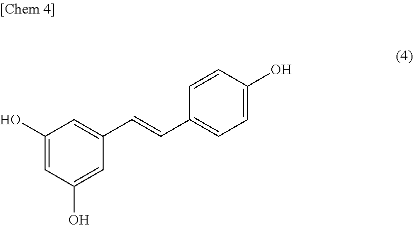 [SMILES: Oc1ccc(/C=C/c2cc(O)cc(O)c2)cc1]

More specifically, the resveratrol can be in the form of a monomer or in the form of an oligomer including up to 4 monomer units (namely an oligomer including up to 4 times the repetition of the molecule of resveratrol). This is why, in the context of the present disclosure, the term «resveratrol» designates both the monomeric form (with the 2 cis and trans isomeric forms) and the oligomeric form of resveratrol without distinction.

The resveratrol is known for its anti-aging properties.

Furthermore, there are derivatives of resveratrol. More specifically, it is resveratrol in the form of monomers or oligomers in which at least one hydroxyl group has been esterified or etherified.

Indeed, the hydroxyl groups of resveratrol are responsible for the air and light instability of this molecule and for its water-soluble character. This is why the protection of the hydroxyl groups of resveratrol by an ester group or an ether group is particularly beneficial, since it gives it satisfactory stability and makes it liposoluble (which also has the advantage of overcoming the problems of miscibility with many cosmetic excipients). Furthermore, these protective ester and ether groups are easily eliminated in vivo by cutaneous enzymes during the application of the cosmetic composition to the skin or skin appendages so as to «release» the resveratrol molecule which will then exert its anti-aging activity on the skin or skin appendages.

### SUMMARY

Nonetheless, there is still a need to find new combinations of molecules which are perfectly effective, or even more effective than those which already exist, for combating the signs of aging of the skin or skin appendages.

Quite unexpectedly, the inventors of the present disclosure have discovered a synergistic effect between the honokiol and magnolol molecules present in magnolia and resveratrol or one of its derivatives to combat certain skin or skin appendages aging signs. Indeed, the inventors have discovered that the application to the skin or skin appendages of a cosmetic composition comprising the combination of these different molecules had an anti-aging effect greater than the cumulative anti-aging effects of honokiol and/or magnolol on the one hand and resveratrol and/or one of its derivatives on the other hand.

This is why the present disclosure has as its first object a cosmetic composition which is characterized in that it comprises at least the following molecules:
i) honokiol and/or magnolol,
ii) resveratrol and/or a derivative of resveratrol.

Like resveratrol, the resveratrol derivative can be in the form of a monomer as well as an oligomer.

Preferably, the resveratrol and the resveratrol derivative that said cosmetic composition comprises, correspond to the following chemical formula (5):

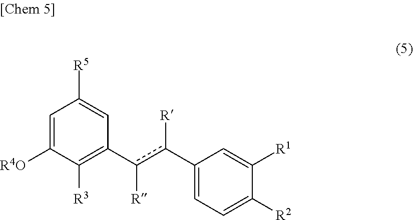

in which
   R′ is a hydrogen or the junction point at R³ or at R⁴ of another monomer unit,
   R″ is a hydrogen or the junction point at R³ or at R″ of another monomer unit,
   R¹ is a hydrogen, an alcohol function, an ether group or an ester group,
   R² is a hydrogen, an alcohol function, an ether group or an ester group,
   R³ represents a hydrogen or the junction point at R″ or R′ of another monomer unit,
   R⁴ is a hydrogen, an alkyl radical, an acyl radical or a junction point at R′ of another monomer unit,
   R⁵ is a hydrogen, an alcohol function, an ether group or an ester group,
and the diastereoisomers and regioisomers of these monomer units.

Preferably, for the reasons explained above as regards the instability due to the hydroxyl groups of resveratrol, the derivative of resveratrol is a resveratrol in which all the hydroxyl groups have been esterified or etherified. This resveratrol derivative is therefore stable and liposoluble.

Preferably, the resveratrol derivative is selected from resveratrol esters which may be in the form of monomers or oligomers, the monomers comprising at least one ester group of the following chemical formula (6):

### [Chem 6]

—O—CO-A (6)
in which A is a linear or branched, saturated or unsaturated, alkyl radical of at least two carbon atoms, an aryl, aralkyl or aralkylene radical,
and the oligomers being formed of:
   monomer units joined by carbon-carbon or ether bonds, and or
   monomer units crosslinked by groups whose chemical formula (7) is as follows:

### [Chem 7]

—O—CO—R—CO—O— (7)
in which R is a saturated or unsaturated alkylene radical having 0 to 10 carbon atoms, an arylene radical having 1 to 3 rings or a heterocyclic radical, and the diastereoisomers of these monomer units.

Advantageously, A is a saturated or unsaturated fatty acid radical. The fatty acid can be selected from butyric, valeric, hexanoic, sorbic, lauric, palmitic, stearic, oleic, linoleic, linolenic, a linolenic, arachidonic, eicosapentaenoic and docosahexaenoic acids. Most preferably, the fatty acid is C16. In other words, A is preferably a C₄-C₂₈, more preferably C₄-C₂₂ and even more preferably C₁₆ alkyl radical.

### DESCRIPTION OF THE DISCLOSURE

In another embodiment of the disclosure, A is an aryl radical, for example phenyl.

In another embodiment of the disclosure, A is an aralkyl or aralkylene radical, the aralkyl or aralkylene radical preferably being C1 to C8, more preferably C1 to C4. For example, it can be the benzyl or styryl radical.

Thus, in these resveratrol derivatives in the form of esters, at least one hydroxyl group has been esterified. As explained above, protecting the hydroxyl groups of resveratrol with an ester group stabilizes resveratrol and makes it liposoluble.

Furthermore, these ester groups are easily eliminated in vivo by cutaneous enzymes, when the cosmetic composition is applied to the skin or skin appendages, so that the skin benefits in a combined manner from the anti-aging properties of resveratrol thus found (because without protection) and moisturizing properties of the acid part of the ester, for example when it is a fatty acid.

Resveratrol derivatives in the form of esters are for example described in the application WO 99/03816 A1.

The resveratrol and the resveratrol derivative can for example be selected from rhapontine, deoxyrhapontine, piceatannol, astringine, pterostilbene, pinosylvine, piceide, viniferins, miyabenol C, pallidol, hopeaphenol and E-vitisin-B. In one embodiment of the disclosure, it is ε-viniferin which is a dimer of resveratrol.

Preferably, the resveratrol or the resveratrol derivative is in trans form. For example, it may be trans-resveratrol, trans-rhapontin, trans-desoxyrhapontin, trans-piceatannol, trans-astringine, trans-pterostilbene, trans-pinosylvine, trans-piceid, trans-ε-viniferin and trans-miyabenol C.

The cosmetic composition according to the disclosure may comprise a mixture of resveratrol which is in the form of monomers and/or oligomers and of derivatives of resveratrol which are in the form of monomers and/or oligomers.

The resveratrol and the resveratrol derivative may have been obtained from at least one plant and/or by chemical synthesis. It may be a plant selected from Vitaceae, Umbelliferae, Myrtaceae, Dipterocarpaceae, Cyperaceae, Gnetaceae, Leguminaceae, Grasses, Sericaceae, Haemodoraceae, Musaceae, Polygonaceae, Pinaceae, Cupressaceae, Caesalpiniaceae, Poaceae and Solanaceae.

In one embodiment of the disclosure, the resveratrol and/or the resveratrol derivative present in said cosmetic composition is in the form of at least one plant extract containing it. Preferably, it is a dry plant extract containing resveratrol and/or a resveratrol derivative.

Such a plant extract is obtained by bringing the plant into contact with water and/or an organic solvent. In this way, the monomers and/or oligomers of resveratrol which are present in the plant are solubilized in water and/or the organic solvent. The extraction can be carried out by subjecting the mixture of plant and water and/or organic solvent to a treatment such as microwaves, ultrasounds, maceration-leaching or even supercritical fluids. Advantageously, the plant extract thus recovered can also be subjected to one or more additional extraction steps using an organic solvent (for example ethyl acetate or ethyl ether). Then, the plant extract can be washed and stored in freeze-dried form. When the plant extract is in freeze-dried form, it is a dry plant extract.

The plant extract is an extract of a plant which can be selected from those which have been listed above. Most preferably, the plant extract is an extract of Vitaceae. The plant extract may be a vine extract, more preferably an extract of vine shoots and/or vine stalks. Preferably, it is a dry extract of vine shoots and/or vine stalks containing resveratrol and/or a derivative of resveratrol. Thus, in one embodiment of the disclosure, the resveratrol and/or the resveratrol derivative present in the said cosmetic composition is in the form of at least one dry extract of vine shoots and/or vine stalks containing it.

In one embodiment of the disclosure, the resveratrol derivative present in the cosmetic composition is in the form of a plant extract containing it. More specifically, it is a plant extract containing resveratrol as described above, all or part of the hydroxyl groups of which have then been esterified or etherified by chemical synthesis. The protection of hydroxyl groups by ester or ether groups is perfectly within the reach of those skilled in the art. For example, the preparation of resveratrol derivatives in the form of esters is described in the application WO 99/03816 A1. Preferably, the protection of the hydroxyl groups has been carried out from a dry plant extract which contains resveratrol (or in other words from a freeze-dried extract of resveratrol).

In one embodiment of the disclosure, the resveratrol or the resveratrol derivative has been obtained from microorganisms genetically modified from animal tissues or by bioproduction from plants, in particular from cell suspensions of vine.

Advantageously, honokiol and/or magnolol is present in said cosmetic composition in the form of at least one magnolia extract containing it. Most preferably, the magnolia extract is an extract of magnolia bark. For example, this extract is obtained by implementing a supercritical fluid in order to limit the use of solvents. In one advantageous embodiment of the disclosure, the honokiol and/or the magnolol is present in said cosmetic composition in the form of at least one liquid extract of magnolia containing it, preferably a liquid extract of magnolia bark.

In one embodiment of the disclosure, the cosmetic composition comprises at least:
an extract of magnolia bark containing honokiol and/or magnolol;
an extract of vine shoots and/or vine stalks containing resveratrol and/or a derivative of resveratrol.

In one advantageous embodiment of the disclosure, the cosmetic composition comprises at least:
a liquid extract of magnolia bark containing honokiol and/or magnolol;
a dry extract of vine shoots and/or vine stalks containing resveratrol and/or a derivative of resveratrol.

The mass ratio between on the one hand the resveratrol and/or the resveratrol derivative and on the other hand the honokiol and/or the magnolol can be comprised between 1:10 and 3:2, preferably between 1:2 and 3:1.

Preferably, the cosmetic composition comprises, in mass percentages expressed relative to the total mass of said composition:
between 0.01% and 0.2%, preferably between 0.02% and 0.1% of honokiol and/or magnolol;
between 0.001% and 0.3%, preferably between 0.01% and 0.3% of resveratrol and/or of resveratrol derivative.

When the cosmetic composition comprises a dry plant extract (preferably a dry extract of vine shoots and/or vine stalks) containing resveratrol and/or a resveratrol derivative, the mass percentage of said dry plant extract expressed relative to the total mass of the cosmetic composition can be comprised between 0.01% and 1%, preferably between 0.1% and 1%.

The plant dry extract (preferably a dry extract of vine shoots and/or vine stalks) may comprise between 10% and 60%, preferably between 20% and 50%, of resveratrol (namely both under the form of monomer than of oligomers). Indeed, advantageously, said plant dry extract may have been obtained from several extraction steps so as to increase the concentration of resveratrol (preferably trans-resveratrol) and resveratrol oligomers (preferably ε-viniferin, δ-viniferin and α-viniferin) within said dry plant extract.

In one embodiment of the disclosure, the plant dry extract comprises, in mass percentages expressed relative to the mass of the dry extract:
between 10% and 30% trans-resveratrol;
between 10% and 20% of ε-viniferin.

Furthermore, as explained above, all or part of the hydroxyl groups of resveratrol (namely both in the form of monomer and of oligomers) which the plant extract contains may have been esterified and/or etherified by chemical synthesis so as to obtain resveratrol derivatives both in the form of monomers and of oligomers.

When the cosmetic composition comprises a liquid plant extract (preferably a liquid magnolia bark extract) containing honokiol and/or magnolol, the mass percentage of said liquid plant extract expressed relative to the total mass of the cosmetic composition can be comprised between 0.2% and 3%, preferably between 0.5% and 2%. Preferably, the mass percentage of the honokiol and/or the magnolol expressed relative to the mass of the liquid plant extract can be comprised between 2% and 10%. This mass percentage can for example be 5%.

Thus, in one embodiment of the disclosure, the cosmetic composition comprises:
the resveratrol and/or a resveratrol derivative in the form of a dry extract of vine shoots and/or vine stalks containing it, the percentage by mass of said dry extract expressed relative to the total mass of the cosmetic composition being comprised between 0.01% and 1%, preferably between 0.1% and 1%,
the honokiol and/or magnolol in the form of a liquid magnolia bark extract containing it, the mass percentage of said liquid extract expressed relative to the total mass of the cosmetic composition being comprised between 0.2% and 3%, preferably between 0.5% and 2%.

The cosmetic composition according to the disclosure further comprises excipients suitable for application to the skin or skin appendages of said cosmetic composition.

These are excipients which are perfectly tolerated by the skin and the skin appendages, and which advantageously have a pleasant odour and appearance.

The choice and the amounts of these excipients used in the cosmetic composition according to the disclosure are perfectly within the reach of those skilled in the art.

The cosmetic composition according to the disclosure can be more or less fluid and have the appearance of a white or coloured cream, an ointment, a milk, a lotion, a serum, a paste or even a mousse. The cosmetic composition according to the disclosure can also be in a solid form, for example in the form of a stick.

The cosmetic composition according to the disclosure can be used as a care product and/or as a make-up product and/or as a hygiene product for the skin or skin appendages.

The cosmetic composition according to the disclosure can be in all the dosage forms normally used in the field of cosmetics.

It may for example be in the form of an optionally gelled aqueous or oily solution, an optionally two-phase dispersion of the lotion type, an emulsion obtained by dispersing a fatty phase in an aqueous phase (O/W) or conversely of an aqueous phase in a fatty phase (W/O), or even of a triple emulsion (W/O/W or O/W/O) or of a vesicular dispersion of the ionic and/or non-ionic type.

The cosmetic composition according to the disclosure can be prepared according to the usual methods. For example, when the cosmetic composition according to the disclosure is an emulsion, the mass percentage of the fatty phase can be comprised between 0.5% and 80%, and preferably between 5% and 50%, relative to the total mass of said cosmetic composition.

The oils, emulsifiers and co-emulsifiers used in the cosmetic composition according to the disclosure in emulsion form are selected from those conventionally used in the considered field.

The mass percentage of emulsifier and co-emulsifier can be comprised between 0.1% and 30%, preferably between 0.5% and 20%, relative to the total mass of said cosmetic composition.

The emulsifiers and co-emulsifiers can be selected from fatty acid and glycerin esters such as glyceryl stearate, sucrose esters and phospholipids.

The cosmetic composition according to the disclosure may comprise at least one oil selected from oils of vegetable origin (for example almond oil, apricot oil, grape seed oil, liquid fraction of shea butter, avocado and soy) and synthetic oils (for example isononyl isononanoate, pentaerythrityl tetraoctanoate).

The cosmetic composition according to the disclosure may also comprise fatty substances selected from fatty alcohols (for example cetyl or stearyl alcohol), fatty acids (for example stearic acid) and waxes (for example carnauba wax, ozokerite or bee).

The cosmetic composition according to the disclosure may also comprise at least one adjuvant selected from the usual adjuvants in the field of cosmetics. They may, for example, be hydrophilic or lipophilic gelling agents, hydrophilic or lipophilic active agents, preservatives, antioxidants, solvents, perfumes, fillers, sunscreens, pigments or colouring materials, absorbers of smell, chelating agents and alcohols. These various adjuvants are described in more detail below.

The mass percentages of these various adjuvants are those conventionally used in the field of cosmetics. For example, these mass percentages are comprised between 0.01% and 20% relative to the total mass of said cosmetic composition.

These adjuvants, depending on their nature, can be introduced into the fatty phase, into the aqueous phase or into the lipid vesicles.

In any case, these adjuvants, as well as their quantities, are selected so as not to harm the desired properties of the combination of honokiol and/or magnolol with resveratrol and/or the derivative of resveratrol.

As hydrophilic gelling agents, mention may be made, for example, of carboxyvinyl polymers, acrylic copolymers such as acrylate/alkylacrylate copolymers, polysaccharides, natural gums and clays, and, as lipophilic gelling agents, mention may be made, for example modified clays such as bentones and hydrophobic silica.

As fillers, mention may be made, for example, of polymethyl methacrylate microspheres, acrylate-acrylate copolymer powders, expanded powders such as hollow microspheres and in particular, powders of natural organic materials such as powders of starch, in particular corn, wheat or rice starches, crosslinked or not, such as starch powders crosslinked with octenylsuccinate anhydride, silica, metal oxides such as titanium dioxide or zinc, mica, and mixtures thereof.

The cosmetic composition according to the disclosure may also contain active molecules other than honokiol and/or magnolol and resveratrol and/or the resveratrol derivative. It may for example be at least one compound selected from antioxidants, moisturizing agents, desquamating agents, agents improving the barrier function of the skin, agents promoting cutaneous microcirculation, depigmenting agents, antiglycation agents, nitric oxide synthase inhibitors, agents stimulating the synthesis of dermal or epidermal macromolecules and/or preventing their degradation, agents stimulating the proliferation of fibroblasts and/or keratinocytes or stimulating the differentiation of keratinocytes, muscle relaxants, tightening agents, anti-pollution and/or anti-radical agents, sunscreens, propigmenting agents, anti-inflammatory agents, agents modifying the production of sebum, refreshing agents and mixtures thereof.

As antioxidants, mention may be made, for example, of tocopherol and its esters, in particular tocopherol acetate, ascorbic acid and its derivatives, in particular ascorbyl magnesium phosphate, ascorbyl glucoside and ascorbyl tetraisopalmitate, ferulic acid, serine, ellagic acid, phloretin and mixtures thereof.

The cosmetic composition according to the disclosure may also contain at least one active molecule selected from vitamin C and its derivatives, vitamin E and its derivatives (for example tocopherol acetate).

The mass percentage of these additional active molecules can be comprised between 0.001% and 20%, preferably between 0.01% and 10%, even more preferably between 0.5% and 5%, relative to the total mass of said cosmetic composition.

The disclosure also relates to a cosmetic treatment method comprising a step of applying to the skin or skin appendages of the cosmetic composition according to the disclosure which has been described above.

The cosmetic treatment method aims to prevent or reduce skin signs such as loss of firmness and/or elasticity of the skin, thinning of the skin, wrinkles and fine lines, a dull appearance of the complexion, hyperpigmentation of the skin or heterogeneity of its pigmentation and pigment spots.

According to such a method of the disclosure, the cosmetic composition is applied in an amount sufficient to stimulate the activity of the TET enzymes. The examples of the description describe a method for determining the activity of the TET enzymes and, by resorting to such a method or any other method, it is within the reach of those skilled in the art to define an appropriate amount.

The cosmetic composition according to the disclosure can be applied directly to the skin or skin appendages or, alternatively, to cosmetic carriers of the occlusive or non-occlusive type, which are intended to be applied locally to the skin. For example, it can be masks, wipes or fabrics.

The application of the cosmetic composition according to the disclosure can for example be daily, several times a day or weekly. It can be continued for several days and/or several weeks, or even longer. This application may be continuous or may for example be continued for 1 to 2 months, then resumed after an interruption.

The disclosure also concerns the non-therapeutic cosmetic use of a cosmetic composition as described above and corresponding to any one of the stated characteristics, to stimulate the activity of TET enzymes. As explained above, the stimulation of such an enzymatic activity makes it possible to fight against the hypermethylation of genes, in particular linked to age, and to reduce the signs of aging of the skin and skin appendages.

Other characteristics and advantages of the disclosure are illustrated in the examples which follow.

Furthermore, the disclosure will be better understood with the aid of the detailed description which is set out below with reference to the appended drawing representing, by way of nonlimiting example, experimental results testifying to the synergistic effect of a vine shoot extract with a magnolia bark extract.

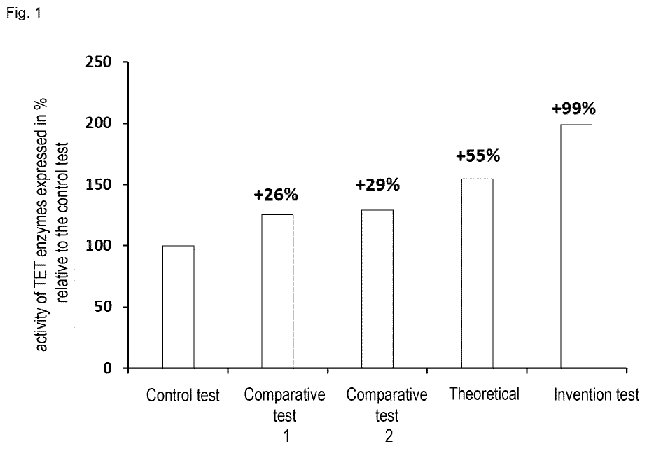

FIG. 1 is a histogram representing the percentage increase in the activity of the TET enzymes of 2 comparative tests and of a test according to the disclosure compared to a control test.

### EXPERIMENTAL PART

### Example 1: Effect of the Combination of a Vine Shoot Extract and a Magnolia Bark Extract on the Activity of TET Enzymes

Primary human fibroblasts were seeded at a cell density of 400,000 cells in 4 Petri dishes with a surface area of 60 cm² of culture medium which contained in a volume of 10 mL:
100 μL of a mixture of penicillin and streptomycin marketed by the company GIBCO under the reference 15140-122, the concentrations of penicillin and streptomycin in the Petri dishes were respectively 100 U/mL and 100 μg/mL;
1 μL of foetal calf serum marketed by the company GIBCO under the reference 16000-044;
Qsp 10 mL of an Eagle medium modified by Dulbecco, known by the abbreviation «D-MEM» which is the acronym for «Dulbecco's Modified Eagle Medium» marketed by the company GIBCO under the reference 11965-092. It is a suitable medium for the growth of mammalian cells which is high in glucose and additionally contains L-glutamine. «Qsp 10 mL» is the abbreviation of «quantity sufficient for 10 mL». This is the volume of Dulbecco's Modified Eagle's Medium that has been added such that the sum of the volumes of this medium, the penicillin/streptomycin mixture, and the foetal bovine serum is 10 mL.

The culture conditions were as follows:
a temperature of 37° C.;
a humidified atmosphere comprising 5% carbon dioxide.

After 24 hours of culture of these primary human fibroblasts:
a liquid magnolia bark extract was added to a 1^{st} petri dish in such an amount that the volume percentage of honokiol and magnolol relative to the volume of the culture medium was 0.001%. This liquid magnolia bark extract comprised in mass percentages expressed relative to the mass of said extract: 5% honokiol and magnolol and 95% propanediol. This petri dish thus constituted a first comparative test.
a dry extract of vine shoots which had been dissolved beforehand in dimethylsulfoxide (known by the abbreviation «DMSO») at a concentration of 6 mg/mL was added to a 2^{nd} petri dish in a quantity such that the volume percentage of resveratrol (namely both in the form of monomer and oligomers) contained in this extract relative to the volume of the culture medium was 0.0013%. This 2^{nd} Petri dish constituted a 2^{nd} comparative test.
a mixture composed of the same liquid extract of magnolia bark used for the 1^{st} comparative test and of the same dry extract of vine shoot used for the 2^{nd} comparative test was added to a 3^{rd} Petri dish, and this in quantities such that the volume percentage of honokiol and magnolol was 0.001% and that of resveratrol (namely both in the form of monomer and of oligomers) was 0.0013% relative to the volume of said culture medium. This 3^{rd} Petri dish constituted a test according to the disclosure.

Moreover, the 4^{th} petri dish in which no plant extract was added to its culture medium constituted the control test.

After 48 hours, the culture media from each of the 4 petri dishes were removed.

For each of the 4 Petri dishes, after washing with a saline phosphate buffer (or in other words a solution containing sodium chloride, disodium phosphate, monopotassium phosphate and potassium chloride), the primary human fibroblasts as well grown were peeled off and counted.

Then, the nuclear proteins present in these primary human fibroblasts thus cultured were extracted using the nuclear protein extraction kit marketed by the company EPIGENTEK under the trade name «EpiQuik™ Nuclear Extraction Kit I». Finally, an assay of these proteins was carried out using a protein assay kit marketed by THERMO SCIENTIFIC under the trade name «Micro BCA™ Protein Assay Kit».

The TET enzymes are part of these extracted and assayed nuclear proteins.

This is why from the nuclear proteins thus extracted and assayed obtained with the 4 tests carried out in the 4 petri dishes detailed above, the activity of the TET enzymes was determined using the evaluation kit of the activity or inhibition of TET enzymes marketed by the company EPIGENTEK under the trade name «Epigenase™ 5mC-Hydroxylase TET Activity/Inhibition Assay Kit».

The measurement of the activity of the TET enzymes was carried out for each test.

Furthermore, all these experiments were repeated 4 times.

Finally, the average of all the measurements of the activity of the TET enzymes was calculated for:
the control test;
the test according to the disclosure;
the 1^{st} comparative test;
the 2^{nd} comparative test.

Table 1 below details relative to the control test:
the activity of the TET enzymes for the 1^{st} comparative test («Comparative test 1») expressed as a percentage relative to the activity of the TET enzymes of the control test;
the activity of the TET enzymes for the 2^{nd} comparative test («Comparative test 2») expressed as a percentage relative to the activity of the TET enzymes of the control test;
the activity of the theoretical TET enzymes corresponding to the sum of the activities of the TET enzymes of the 1^{st} comparative test and of the 2^{nd} comparative test ((«Theoretical») expressed as a percentage relative to the activity of the TET enzymes of the control test;
the activity of the TET enzymes for the test according to the disclosure («Test disclosure») expressed as a percentage relative to the activity of the TET enzymes of the control test.

TABLE 1

| | Compara- | Compara- | | Disclosure |
|---|---|---|---|---|
| | tive test 1 | tive test 2 | Theoretical | test |
| Increase in TET | 26 | 29 | 55 (namely | 99 |
| enzyme activity in % | | | 26 + 29) | |

FIG. 1 represents a histogram of the activity of the TET enzymes of the 1^{st} comparative test, of the 2^{nd} comparative test, of the test according to the disclosure, as well as the activity of the theoretical TET enzymes corresponding to the sum of the activities of the TET enzymes of the 1^{st} comparative test and of the 2^{nd} comparative test, expressed in percentages relative to the activity of the TET enzymes of the control test set at 100.

In view of Table 1 and FIG. 1, it is noted that the activity of the TET enzymes of the test according to the disclosure is 99% and is therefore much higher than:
that of comparative test 1 (namely the test for which only the magnolia extract was used);
that of comparative test 2 (namely the test for which only the vine shoot extract was used).

It should above all be noted that this activity of the TET enzymes is much higher (namely by 44%) than the activity of the theoretical (or in other words expected) TET enzymes of 55% which corresponds to the sum of the activities of the TET enzymes of comparative tests 1 and 2 expressed as percentages relative to the activity of the TET enzymes of the control test. These experiments demonstrate the synergistic effect between magnolia bark extract and vine shoot extract on the activity of TET enzymes.

Thus, these in vitro experiments show that the combination of honokiol and/or magnolol (present in a magnolia bark extract) with resveratrol and/or a resveratrol derivative (present in a vine) has an entirely beneficial synergistic effect for combating the signs of aging of the skin and skin appendages, so as to improve their appearance, and that it is therefore perfectly suitable in an anti-aging cosmetic composition.

### Example 2: Cosmetic Compositions According to the Disclosure

Examples of cosmetic compositions according to the disclosure are detailed below.

Table 2 below is an example of a cosmetic composition according to the disclosure in the form of an emulsion whose constituents are mentioned with their names of the INCI nomenclature (INCI being the abbreviation of «International Nomenclature of Cosmetic Ingredients».

The mass percentages of each of the constituents are expressed relative to the total mass of said composition.

«Qsp 100» is the abbreviation of «quantity sufficient for 100%». This is the mass percentage of water so that the sum of the mass percentages of all the constituents of the cosmetic composition reaches 100%.

«Qsp pH 5-5.5» is the abbreviation of «quantity sufficient for pH 5-5.5». This is the mass percentage of potassium hydroxide so that the pH of the cosmetic composition is comprised between 5 and 5.5.

TABLE 2

| | Mass percentage |
|---|---|
| INCI name of constituents | (%) |
| Aqua | Qsp 100 |
| Glycerine | 10 |
| Butylene glycol | 2 |
| Caprylic/capric triglyceride | 2 |
| Hydrogenated ethylhexyl olivate | 2 |
| Cetearyl alcohol | 1.5 |
| Cetearyl glucoside | 1.5 |
| Hydrogenated olive oil unsaponifiables | 1.5 |
| Vitis vinifera seed oil | 1.5 |
| Coco-caprylate/caprate | 1.5 |
| Sesamum indicum seed oil | 1.5 |
| Butyrospermum parkii butter extract | 1.5 |
| Pentaerythrityl distearate | 1 |
| Potassium cetyl phosphate | 1 |
| Resveratrol oligomer | 0.01 to 5 |
| Caprylyl glycol | 0.2 |
| Perfume | 0.2 |
| Magnolia grandiflora bark extract | 0.01 to 5 |
| Potassium sorbate | 0.1 |
| Tocopherol | 0.054 |
| Sodium hydroxide | Qsp pH 5.00-5.50 |
| Sodium phytate | 0.025 |

Table 3 below is an example of a cosmetic composition according to the disclosure in the form of a serum whose constituents are mentioned with their names from the INCI nomenclature.

TABLE 3

| | Mass percentage |
|---|---|
| INCI name of constituents | (%) |
| Aqua | Qsp 100 |
| Glycerine | 5 |
| Propanediol | 3 |
| Caprylyl glycol | 1 |
| Glyceryl caprylate/caprate | 1 |
| Carbomer | 0.5 |
| Helianthus Annuus seed oil | 0.5 |
| Tocopherol | 0.15 |
| Resveratrol oligomer | 0.01 to 5 |
| Perfume | 0.1 |
| Magnolia grandiflora bark extract | 0.01 to 5 |
| Sodium hydroxide | QSP pH 5.00-5.50 |
| Sodium phytate | 0.025 |

1. A cosmetic composition comprising at least the following molecules:
i) honokiol and/or magnolol,
ii) resveratrol and/or resveratrol derivative.

2. The cosmetic composition according to claim 1, wherein the resveratrol and the derivative of resveratrol correspond to the following chemical formula (5): 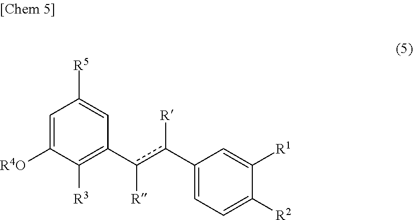
in which:
R′ is a hydrogen or the junction point at R³ or at R⁴ of another monomer unit,
R″ is a hydrogen or the junction point at R³ or at R″ of another monomer unit,
R¹ is a hydrogen, an alcohol function, an ether group or an ester group,
R² is a hydrogen, an alcohol function, an ether group or an ester group,
R³ represents a hydrogen or the junction point at R″ or R′ of another monomer unit,
R⁴ is a hydrogen, an alkyl radical, an acyl radical or a junction point at R′ of another monomer unit, and
R⁵ is a hydrogen, an alcohol function, an ether group or an ester group, and the diastereoisomers and regioisomers of these monomer units.

3. The cosmetic composition according to claim 1, wherein the resveratrol derivative is selected from resveratrol esters in the form of monomers or oligomers, the monomers comprising at least one ester group of the following chemical formula:
[Chem 6]
—O—CO-A (6)
in which A is a linear or branched, saturated or unsaturated alkyl radical of at least two carbon atoms, an aryl, aralkyl or aralkylene radical,
and the oligomers being formed of:
monomer units joined by carbon-carbon or ether bonds, and or
monomer units cross-linked by groups whose chemical structure is as follows:
[Chem 7]
—O—CO—R—CO—O (7)
in which R is a saturated or unsaturated alkylene radical having 0 to 10 carbon atoms, an arylene radical having 1 to 3 rings or a heterocyclic radical, and the diastereoisomers of these monomer units.

4. The cosmetic composition according to claim 3, wherein A is a saturated or unsaturated fatty acid radical which is selected from the group consisting of: butyric, valeric, hexanoic, sorbic, lauric, palmitic, stearic, oleic, linoleic, linolenic, α linolenic, arachidonic, eicosapentaenoic, and docosahexaenoic.

5. The cosmetic composition according to claim 1, wherein the resveratrol and the resveratrol derivative are selected from the group consisting of: rhapontine, deoxyrhapontine, piceatannol, astringine, pterostilbene, pinosylvine, piceide, viniferins, miyabenol C, pallidol, hopeaphenol and E-vitisin-B.

6. The cosmetic composition according to claim 1, wherein the composition further comprises a mixture of resveratrol which is in the form of monomers and/or oligomers and of resveratrol derivatives which are in the form of monomers and/or oligomers.

7. The cosmetic composition according to claim 1, wherein the resveratrol and the resveratrol derivative have been obtained from at least one plant and/or by chemical synthesis.

8. The cosmetic composition according to claim 7, wherein the plant is selected from the group consisting of: Vitaceae, Umbelliferae, Myrtaceae, Dipterocarpaceae, Cyperaceae, Gnetaceae, Leguminaceae, Gramineae, Sericaceae, Haemodoraceae, Musaceae, Polygonaceae, Pinaceae, Cupressaceae, Caesalpiniaceae, Poaceae, and Solanaceae.

9. The cosmetic composition according to claim 1, wherein the resveratrol and/or the resveratrol derivative present in the said cosmetic composition is in the form of at least one dry extract of vine shoots and/or vine stalks containing it.

10. The cosmetic composition according to claim 1, wherein honokiol and/or magnolol is present in the said cosmetic composition in the form of at least one liquid magnolia extract containing it.

11. The cosmetic composition according to claim 10, wherein the liquid magnolia extract is a liquid extract of magnolia bark.

12. The cosmetic composition according to claim 1, wherein the mass ratio between, on the one hand, resveratrol and/or the resveratrol derivative and, on the other hand, honokiol and/or magnolol is comprised between 1:10 and 3:2.

13. The cosmetic composition according to claim 1, wherein it comprises, in mass percentages expressed relative to the total mass of said composition:
between 0.01% and 0.2% of honokiol and/or magnolol; and
between 0.001% and 0.3% of resveratrol and/or of resveratrol derivative.

14. The cosmetic composition according to claim 10, wherein it comprises:
resveratrol and/or a resveratrol derivative in the form of a dry extract of vine shoots and/or vine stalks containing it, the percentage by mass of said dry extract expressed relative to the total mass of the cosmetic composition being comprised between 0.01% and 1%, and
honokiol and/or magnolol in the form of a liquid magnolia bark extract containing it, the mass percentage of said liquid extract expressed relative to the total mass of the cosmetic composition being comprised between 0.2% and 3%.

15. A cosmetic treatment method including a step of applying the cosmetic composition to the skin or the skin appendages according to claim 1.

16. The method according to claim 15, wherein the cosmetic composition is in a sufficient amount to stimulate the activity of the TET enzymes.

17. A non-therapeutic cosmetic use of a cosmetic composition according to claim 1, for stimulating the activity of TET enzymes.
